(19) United States
(12) Patent Application Publication
(10) Pub. No.: US 2014/0154685 A1
(43) Pub. Date: Jun. 5, 2014

(54) METHOD AND KIT FOR MEASURING MICRO-RNA IN BODY FLUIDS

(71) Applicant: Rosalind Franklin University Of Medicine And Science, (US)

(72) Inventor: Dominik M. DUELLI, Lake Bluff, IL (US)

(73) Assignee: Rosalind Franklin University Of Medicine And Science, North Chicago, IL (US)

(21) Appl. No.: 13/692,477

(22) Filed: Dec. 3, 2012

(51) Int. Cl. C12Q 1/68

(52) U.S. Cl. CPC C12Q 1/686
USPC 435/6.12

(57) ABSTRACT

The present invention provides a kit for measuring the content of micro-RNA in a human blood sample including a blood collection tube containing at least 1 μg NaF and 0.8 μg KOx; and providing a set of instructions for collecting a human blood sample in the blood collection tube.

### FEDERALLY SPONSORED RESEARCH

This work is supported by U.S. Army Medical Research and Materiel Command under W81XWH-08-1-0641, NIH grant No. R01 CA119903 and NIH grant No. T32-AI007392/AI/NIAID NIH HHS.

### BACKGROUND OF THE INVENTION

1. Technical Field

The present invention provides a method and kit for measuring the quantity of microRNAs in human body fluids more accurately than prior art methodologies.

2. Background of the Invention

Circulating microRNAs (miRNAs) have emerged as candidate biomarkers of various diseases and conditions including malignancy and pregnancy. This approach requires sensitive and accurate quantitation of miRNA concentrations in body fluids. Enzyme-based miRNA quantitation, which is currently the mainstream approach for identifying differences in miRNA abundance among samples, is skewed by endogenous serum factors that co-purify with miRNAs, and anticoagulants used during collection. What is important, different miRNAs were affected to different extents in different patient samples. The present invention provides a method and kit that overcome these interfering activities to increase the accuracy and sensitivity of miRNA detection in human body fluids up to 30-fold.

MicroRNAs are small, non-coding RNA sequences of about 19-22 nucleotides that function in modulating the activity of specific mRNA targets in development, differentiation, or disease, typically by compromising mRNA stability or interfering with translation (reviewed in¹). Recently, miRNAs circulating in body fluids, and blood serum in particular, have emerged as promising markers of disease and other processes (reviewed in¹⁻⁵). Accordingly, it is important to accurately identify and quantitate miRNAs in samples collected from patients.

The mainstream approach to identify and quantify miRNAs, utilizes quantitative reverse-transcriptase polymerase chain reaction (qRT-PCR). Using qRT-PCR, changes in plasma and serum miRNA profiles have been reported to reflect various physiological and pathological conditions, including diagnostic and prognostic value for colorectal cancer, breast cancer, gastric cancer, leukemia, lung cancer, lymphoma, oral cancer, ovarian cancer, pancreatic cancer, prostate cancer (reviewed in¹⁻⁵) and other diseases or conditions⁶⁻⁹. The widening use of cell-free circulating miRNA for diagnostic and prognostic purposes, as for any such marker, requires assurance that the measured concentration represents the actual amounts in the samples. Such assurances are often lacking¹⁰. The problem is exacerbated by the common assumption that a protocol developed for one study is applicable for others¹¹. Overall, few methods¹² and improvements¹³ have been offered¹⁴, and commonly used approaches have been shown to lack required accuracy¹⁵. Circulating microRNAs (miRNAs) have emerged as candidate biomarkers of various diseases and conditions including malignancy and pregnancy. This approach requires sensitive and accurate quantitation of miRNA concentrations in body fluids. Here we report that enzyme-based miRNA quantitation, which is currently the mainstream approach for identifying differences in miRNA abundance among samples, is skewed by endogenous serum factors that co-purify with miRNAs, and anticoagulants used during collection. Importantly, different miRNAs were affected to different extents in different patient samples. By developing measures to overcome these interfering activities, we increase the accuracy, and improve the sensitivity of miRNA detection up to 30-fold. Overall, our study outlines key factors that prevent accurate miRNA quantitation in body fluids and provides approaches that allow faithful quantitation of miRNA abundance in body fluids.

### INTRODUCTION

MicroRNAs (miRNAs) are small non-coding RNA sequences of about 19-22 nucleotides that function in modulating the activity of specific mRNA targets in development, differentiation, or disease, typically by compromising mRNA stability or interfering with translation (reviewed in¹). Recently, miRNAs circulating in body fluids, and blood serum in particular, have emerged as promising markers of disease and other processes (reviewed in¹⁻⁵). This application raises the need to accurately identify and quantitate miRNAs in samples collected from patients.

The mainstream approach to identify and quantify miRNAs, utilizes quantitative reverse-transcriptase polymerase chain reaction (qRT-PCR). Using qRT-PCR, changes in plasma and serum miRNA profiles have been reported to reflect various physiological and pathological conditions, including diagnostic and prognostic value for colorectal cancer, breast cancer, gastric cancer, leukemia, lung cancer, lymphoma, oral cancer, ovarian cancer, pancreatic cancer, prostate cancer (reviewed in¹⁻⁵) and other diseases or conditions⁶⁻⁹. The widening use of cell-free circulating miRNA for diagnostic and prognostic purposes, as for any such marker, requires assurance that the measured concentration represents the actual amounts in the samples. Such assurances are often lacking¹⁰. The problem is exacerbated by the common assumption that a protocol developed for one study is applicable for others¹¹. Overall, few methods¹² and improvements¹³ have been offered¹⁴, and commonly used approaches have been shown to lack required accuracy¹⁵.

In this study our goal was to standardize and optimize miRNA detection for biomarker studies. We quantified two miRNAs that are implicated in distinct processes. One was miR-16, which acts as a tumor suppressor, is UV-inducible, p53-regulated and is deregulated or lost in some cancers (reviewed in¹⁶). MiR-16 has also been used to normalize quantitation of circulating miRNAs for breast cancer studies¹⁷⁻¹⁹. The second miRNA, miR-223, has been implicated in pregnancy, other conditions and malignancy^{6,20,21}. Devising reliable approaches for accurate quantitation of circulating miRNAs is important in order to assess their potential as biomarkers.

### BRIEF DESCRIPTION OF THE DRAWINGS

FIGS. 1A,B Fluoride and Oxalate Increase the Quantitation Efficiency of Circulating MicroRNAs. FIG. 1A MiR-16 detection is affected by blood collection method. Plasma and sera were collected from 4 individuals on 3 separate occasions. MiR-16 was amplified using RT-PCR and separated by PAGE. Arrow: miR-16 as determined by sizing and sequencing. FIG. 1B Collection of blood in the presence of EDTA, citrate or fluoride and oxalate increases microRNA detection. MiR-16 and miR-223 were quantified using SYBR green qRT-PCR approaches. Data were analyzed using one-way analysis of variance (ANOVA) followed by Tukey's HSD test. The middle bar represents the median, and the top and bottom of the box are the 75th and 25th percentile. The Tukey fences indicate the range of the values. P values are labeled above or below brackets. Chemicals listed indicate the coagulant and/or blood stabilization agents present in the particular VACUTAINER® used for the blood collection (Materials and Methods). Heparinase indicates the addition of heparinase I to the sample (Materials and Methods). Lower panel. MiR-223 detection is robust in NaF/KOx. Detection of miR-16 or miR-223 was less in heparinized plasma than serum. Heparinase treatment did not always improve detection of miR-16 or miR-223 in heparinized plasma (p=0.082 or p=0.125 respectively). 50 bp and 60 bp indicate the DNA length of the bands in the marker.

FIGS. 2A-C. Fluoride and Oxalate Improve Detection of RNA Regardless of Origin. FIG. 2) NaF/KOx was added at indicated concentrations into 50 μl of serum and plasma collected into EDTA. MiR-16 detection at both concentrations was increased (p=0.014 for both concentrations in serum, and p=0.037 for both concentrations in EDTA plasma. There was no statistical difference between the two concentrations of NaF/KOx, p=0.827 for serum, and p=0.275 for EDTA plasma), (n=3). FIG. 2B Synthetic RNA (SYNTH was added at 250 fmoles per μl during the addition of Trizol to the serum samples or plasma collected using indicated additives. The effectiveness of SYNTH quantitation was compared to that of endogenous miR-16. FIG. 2C) The amplification efficiency of miR-16 and SYNTH in qPCR was measured and compared to that of serum (miR-16: Heparin: p=0.58, Heparin+Heparinase: p=0.43, EDTA: p=0.41, Citrate: p=0.52, KOx: p=0.43, SYNTH: Heparin: p=0.53, Heparin+Heparinase: p=0.49, EDTA: p=0.63, Citrate: p=0.41, KOx: p=0.47, n=3).

FIGS. 3A-E. Removal of Blood Plasma and Serum Components That Inhibit Detection of Circulating MiRNA Using Organic Extraction and Silica-Based RNA Enrichment. FIG. 3A Particulate miR-16 released from cells and added to plasma or whole blood is not detectable within 17 hours. Particulate RNAs released from BC3 cells was incubated in PBS, plasma or blood for indicated time, and miR-16 was quantified by TaqMan qRT-PCR. Portions of miR-16 in 2 h blood are likely of whole-blood origin. FIG. 3B Phenol/chloroform extraction and silica absorption of RNA remove inhibitors of miRNA detection. RNA was isolated from plasma collected in EDTA using Trizol only (left panel), or Trizol followed by one extraction of phenol/chloroform and enrichment of small RNAs on a silica membrane (right panel), and cDNA was added to a standard PCR reaction to quantify SYNTH RNA. RNA was added at the same concentration used in a standard qRT-PCR reaction (indicated by a “1” above the gel), and 10-fold dilutions thereof (10⁻¹, 10⁻², 10⁻³). FIG. 3C Enhanced detection of miR-16 in plasma extracted with phenol/chloroform and silica absorption. Plasma preparations were extracted using indicated numbers of phenol chloroform extractions, followed by absorption to silica, or directly assessed by qRT-PCR. FIGS. 3D, E Low volumes of plasma or serum effect greater detection of miRNAs. Indicated volumes of serum extracted by Trizol were subjected to end-point PCR for miR-16 and separated by PAGE. Filled circles indicate amplification-independent products. MiR-16 was quantified in EDTA plasma immediately after collection, or after storage at −80° C. SYBR Green or TaqMan.

FIGS. 4A-C. Heparin Interference with Detection of MiRNA is Relieved by Heparinase Treatment. Plasma was collected in VACUTAINER® blood tubes containing sodium heparin, and indicated volumes of plasma were assessed by end-point PCR. FIG. 4A. RT-PCR was performed after treatment with Heparinase I (+) or on RNA without heparinase treatment (−). PCR products for miR-16 FIG. 4B and miR-223 FIG. 4C were assessed by PAGE.

FIGS. 5A-D. Mutant Taq DNA Polymerase, Hemo KlenTaq Improves the Sensitivity of MicroRNA Detection. FIG. 5A Hemo KlenTaq (HK) was used to detect miR-16 (arrows) in 200 μl, 50 μl and 10 μl of plasma and serum used in FIG. 3C. Additional PCR products are marked with a star. FIG. 5B Quantitative TaqMan PCR of dilutions of reverse-transcribed miR-16 in serum or NaF/KOx plasma, and using an intact Taq polymerase (i) plus Hemo KlenTaq (HKi) as indicated. FIG. 5C PAGE of miR-16 amplified with HKi or i from 6 individuals (1-6) and FIG. 5D absolute quantitation of miR-16 amplification products. ** indicates P<0.01, *** indicates P<0.001 by Tukey-Kramer Multiple Comparisons test.

FIGS. 6A,B Screening of Taq and non-Taq DNA Polymerases in Serum-Derived Samples. FIG. 6A The functionality of GoTaq, Phire, Phusion and Hemo KlenTaq were tested using cDNA of miRNA isolated from the indicated amount of starting volume of serum by endpoint PCR (40 cycles). (−) indicates PCR reaction was performed with Phire (left lane) or GoTaq (right lane) but with no template. FIG. 6B Quantitation by TaqMan. Detection of miR-16 was successful only for reactions using GoTaq.

FIG. 7. Components in Blood-Derived cDNA Produce Spurious PCR Amplification Products. Hemo KlenTaq was used to amplify miR-16 cDNA produced from miRNAs released from BeWo cells into tissue culture media (BeWo exosomes). The cDNA was supplemented with dilutions of plasma-derived cDNA (Blood cDNA (+)), or without additional cDNA. Copy-DNA products were used unpurified (1), or subjected to purification using ethanol precipitation (2), or a PCR clean kit (3).

FIGS. 8A,B. Titration of Hemo Klentaq With an Intact Taq Polymerase in TaqMan and SYBR Green Reactions Improves PCR Yield. FIG. 8A PAGE analyses of end-point PCR products of reactions supplemented with indicated volume of Hemo KlenTaq in TaqMan or SYBR Green mastermixes as indicated. FIG. 8B, Quantitative PCR analyses of these reactions. Addition of 0.2 μl of Hemo KlenTaq to a 20 μl PCR reaction containing intact Taq polymerase was sufficient to amplify specific PCR products suitable for qPCR by SYBR Green or TaqMan with increased amplification efficiency.

FIGS. 9A,B Hemo KlenTaq Improves Detection of miR-16 in Plasma and Serum Samples. FIG. 9A Serial dilutions of oligos synthesized to reflect miR-16 cDNA product during standard RT was quantified and plotted using GoTaq alone or GoTaq and Hemo KlenTaq. GoTaq/Hem KlenTaq amplification efficiency: 1.07+/−0.06 template duplications/cycle. Go Taq: 0.92+/−0.01 template duplications/cycle). FIG. 9B Average cycle number at threshold. Samples 1-6 are the same as in FIG. 5D.

### DETAILED DESCRIPTION OF THE INVENTION

While this invention is susceptible of embodiment in many different forms, there is shown in the drawings, and will be described herein in detail, specific embodiments thereof with the understanding that the present disclosure is to be considered as an exemplification of the principles of the invention and is not intended to limit the invention to the specific embodiments illustrated.

The present invention provides a method and a kit to standardize and optimize miRNA detection for biomarker studies. We quantified two miRNAs that are implicated in distinct processes. One was miR-16, which acts as a tumor suppressor, is UV-inducible, p53-regulated and is deregulated or lost in some cancers (reviewed in¹⁶). MiR-16 has also been used to normalize quantitation of circulating miRNAs for breast cancer studies¹⁷⁻¹⁹. The second miRNA, miR-223, has been implicated in pregnancy, other conditions and malignancy^{6,20,21}.

In one preferred form of the invention, a kit is provided for measuring the content of micro-RNA in a human blood sample. The kit includes a blood collection tube for collecting, for example, 5 ml of human blood. Blood collection tubes have colored tops that are standardized by all manufacturers. Green tops designate the tube contains sodium or lithium heparin, purple or lavender topped tubes contain EDTA, gray-topped tubes contain sodium fluoride and potassium oxalate and light-blue topped tubes container sodium citrate. Commercially available VACUTAINER® blood collection tubes having gray closures contain 0.5 μg/μl and 0.4 μg/μl KOx.

In one preferred kit of the present invention, the quantity of NaF will be from about 1.0 μg/μl to about 4.0 μg/μl and the quantity of KOx will be from about 0.8 μg/μl to about 3.2 μg/μl in the blood collection tube. The NaF and KOx can be present in the tube prior to taking a blood sample or added to the tube after the blood sample has been taken. The kit will include a blood collection tube, a quantity of NaF and KOx in the ranges set forth above and a set of instructions for using the kit. The kit will be packaged in a container appropriate for its purpose.

In another preferred form of the invention, a kit is provided for use with a gray-topped blood collection container. The kit can optionally include a gray-topped blood collection tube. The gray-topped tube is optional as the kit can be used with a blood sample that has already been taken in a standard gray-topped blood collection tube. The kit will also include a quantity of NaF and KOx to be added to the gray-topped tube to increase the quantity of NaF and KOx to be within the desired ranges set forth above and preferably from about 0.5 μg/μl to about 3.5 μg/μl and the quantity of KOx will be from about 0.4 μg/μl to about 2.8 μg/μl. The NaF and KOx can be in separate containers, combined together in the same container or can be added to the gray-topped container prior to packaging. The NaF and KOx can be in powder form or in separate tablets or in a single tablet. The kit will also include a set of directions for using the kit with existing samples or with blood samples to be taken and will be packaged in suitable packaging.

In another preferred form of the invention, a kit is provided for use with heparin containing blood samples such as the green-topped blood collection containers. This kit will include a quantity of NaF and KOx in the desired amounts set forth above and in the desired powdered or tableted form. The kit will also include a quantity of heparinase I in powder or tableted form to digest the heparin present in the blood sample. The kit will include an appropriate set of instructions for using the kit and will be packaged in suitable medical packaging.

The blood sample can be whole blood, serum or plasma.

The present invention further provides a method for collecting blood from a human subject for measuring the quantity of miRNA in the sample. The method includes the steps of: (1) providing a sample of human blood in a blood collection tube; (2) adding a quantity of NaF and KOx to the blood collection tube so that the blood collection tube has at least 1 μg NaF and 0.8 μg KOx; (3) extracting miRNA from the blood collection tube; and (4) quantifying the miRNA in the sample. In a preferred form of the invention, the human blood sample will have a volume of 5 ml and can be whole blood, serum or plasma.

A preferred method for quantifying the miRNA is through a polymerase chain reaction procedure and particularly one using a combination of Hemo KlenTaq and an intact Taq polymerase. In one preferred form of the invention, the Hemo KlenTaq was present in an amount by volume of from about 2% to about 8% of the volume of the PCR sample.

Materials and Methods

Fresh blood samples (5 ml) were collected from healthy adults, or received from the Susan G Komen Foundation for the Cure Tissue Bank at the IU Simon Cancer Center (SGK samples), or supplied by Dr. Jeffrey Martin from the AIDS Cancer and Specimen Resource (AIDS samples), San Francisco, Calif. Blood was collected in BD VACUTAINER® tubes containing heparin (sodium heparin, 143 usp unit, 10 ml), EDTA (EDTA 7.2 mg, 4.0 ml), sodium citrate (sodium citrate, 0.105 M, 4.5 ml), or sodium fluoride and potassium oxalate (sodium fluoride/potassium oxalate, 5 mg/4 mg, 2 ml). Serum (7.5 ml) was collected in BD SSTT™, BD VACUTAINER®. Plasma was separated from red blood cells promptly to prevent loss of components²² or hemolysis²³. Blood was allowed to coagulate for 15 min at room temperature prior to prompt centrifugation. All donors provided written consent and the work was approved by the RFUMS IRB under protocols #004 and #005 PATH.

Processing of Blood Samples

Fresh plasma and serum were obtained by centrifugation of blood samples at 200 g for 15 min at 4° C. Supernatants were removed and collected in 15 ml polypropylene tubes. The plasma was centrifuged twice at 800 g for 15 min at 4° C. to obtain cell-free plasma. After the second centrifugation, supernatants were collected and passed through 0.45 μm pore-size filters (PALL, Port Washington, N.Y.). Plasma and serum samples were divided into 200 μl, 50 μl and 10 μl samples, and total volumes were adjusted to 200 μl using water. A synthetic RNA, (SYNTH, formerly INT¹⁸, 250 fmol/μl) was added and samples were analyzed immediately or flash-frozen.

Heparinase Treatment of Samples

Digestion of plasma samples with Heparinase I (Sigma-Aldrich, St. Louis, Mo.; H2519) was performed according to the manufacturer's protocol as follows. Heparinase I (55 units) was dissolved at 1 mg/ml in 20 mM Tris-HCl, pH 7.5, 50 mM NaCl, 4 mM CaCl₂, and 0.01% BSA and added to a final concentration of 143 usp units per 10 ml of sample, which is expected to nearly completely remove heparin²⁴. All digestions were performed for 1 h at room temperature, and heparinase was removed using phenol-chloroform extractions.

Post-Collection Treatment of Samples with Sodium Fluoride and Potassium Oxalate

Potassium oxalate (Aqua Solutions, Deer Park Tex.; P5311, KOx) and sodium fluoride (Sigma-Aldrich, S-6776, NaF) were used for these experiments. Five ml blood was collected in NaF/KOx VACUTAINER® blood Tubes® containing 5 mg of NaF and 4 mg of KOx, effecting a final concentration of 1 μg/μl of NaF and 0.8 μg/μl KOx (1×). Therefore, 50 μl of serum and EDTA samples were supplemented with NaF/KOx at 8× (8 μg/μl NaF/6.4 μg/μl KOx), 4× (4 μg/μl NaF/3.2 μg/μl KOx), 2× (2 μg NaF/1.6 μg/μl KOx), 1× (1 μg/μl NaF/0.8 μg/μl KOx), and 0.5× (0.5 μg/μl NaF/0.4 μg/μl KOx) as indicated.

Determination of Exosomal miRNA Stability in Blood Plasma and Whole Blood

Blood was collected in EDTA BD VACUTAINER® blood Tubes®, and half was processed to produce cell-free plasma, the other half was left intact. To test the stability in blood of miRNAs released from cells in culture, aliquots of the plasma and serum were supplemented with exosomal miRNA of BC3 cells, and incubated for the indicated amount of time at 10° C. in a continuously revolving tube rotator.

RNA Isolation

SDS was added to 200 μl samples of plasma/serum for a final concentration of 0.5% where indicated. This preparation was extracted with 500 μl or 1 ml of Trizol LS reagent (Invitrogen) and incubated for 10 minutes at room temperature followed by 100 or 200 μl of chloroform. The mixture was centrifuged at 12,000 g for 16 min and the aqueous layer was transferred into a new tube. Where indicated, this preparation was extracted with acidic phenol/chloroform (Amresco, Solon, Ohio; 0966) one to three times. The resulting aqueous phase was transferred into a new tube and applied toPureLink™ miRNA isolation kit (Invitrogen, Carlsbad, Calif.) as indicated and processed according to the manufacturer's recommendations. RNA was eluted with 50 μl of RNase-free water and stored at −70° C. or used immediately.

Cell Culture

Exosomes and other particulates were collected from cells in culture as described¹⁸. In brief, after 5 days of culturing, the media was collected, centrifuged at 300 g for 15 min, and filtered through a 0.45 μm filter to remove cell debris. The supernatant was centrifuged at 70,000 g to collect particulates including exosomes, and resuspended with 100 μl PBS. BeWo cells were purchased from ATCC (CCL-98, Manassas, Va., USA) and cultured in F-12K or RPMI 1640 (Mediatech Inc. Manassas, Va. or Hyclone Logan, Utah respectively) with 10% or 20% fetal bovine serum (FBS) respectively. To remove bovine particulates, including exosomes, FBS was ultracentrifuged at 70,000 g for 2 h, and the collected supernatant was added to culture media. BC3 cells were cultured as described²⁵.

Reverse Transcription (RT)

For studies with fresh plasma, serum and SGK samples, 10 μl of the 50 μl extracted RNA was used as input into a Superscript III (Invitrogen, Carlsbad, Calif.) reverse transcriptase reaction with miRNA-specific stem-loop primers in the Duelli laboratory as described^{18 26}. The thermal cycles used to amplify the samples was 65° C. for 5 min, 50° C. for 60 seconds, 70° C. for 15 seconds for 40 cycles. AIDS samples and samples including miRNAs released from BC3 cells were analyzed using primers from Applied Biosystems, Inc., ABI (Foster City, Calif.), also using miRNA-specific stem-loop primers in the Cullen laboratory as described^{26 27}.

MicroRNA Quantitation by Taq-Based PCR

Quantitative PCR reactions were performed as described using SYBR® Green or TaqMan® (ABI) as noted¹⁸. Four percent of the cDNA produced in the RT reaction was amplified in MicroAmp™ Optical 96-well reaction plates in triplicate 20 μl reactions on an Applied Biosystems 7900HT Thermocycler (ABI) using the cycle 95° C., 10 min; 40 cycles of 95° C., 15 sec and 60° C. for 1 min; and hold at 4° C. Raw data was analyzed with SDS Relative Quantitation Software version 2.2.3 (ABI), generally using the automatic cycle threshold (Ct) setting for assigning baseline and threshold for Ct determination. MiRNA abundance was measured by computing amoles based on comparing CT values of samples to dilutions of a synthetic DNA corresponding to the cDNA produced by RT for each miRNA measured to make a standard curve. The amplification efficiency, a measure of number of template duplications per PCR amplification cycle was calculated using the equation (T2/T1)^{(1/(CT2 ave-CT1 ave))}−1²⁸.

PCR with Other Polymerases

GoTaq® Green (Promega, Madison, Wis.) PCR was performed in 25 μl volumes according to manufacturer's instructions. The initial denaturation step was 5 min at 95° C., followed by 40 cycles of 15 seconds at 95° C., 30 seconds at 50° C., 30 seconds at 72° C. and a final extension of 5 min at 73° C.

Hemo KlenTaq™²⁹ (New England BioLabs, Ipswich, Mass.) PCR¹⁸ was performed in 25 μl volumes according to manufacturer instructions, including attempts to reduce non-specific priming by assembling PCR reactions on ice and transfer of reactions to the thermocycler preheated to 95° C. PCR using cocktails containing GoTaq® DNA polymerase and Hemo KlenTaq™ polymerase were performed using GoTaq® qPCR Mastermix (Promega) for SYBR Green quantitation or TaqMan® Universal PCR Mastermix (ABI).

Phire and Phusion enzymes were used according to supplier specifications (New England Biolabs) for end-point PCR, or used with GoTaq® qPCR Mastermix (Promega) for SYBR Green or with TaqMan Universal PCR Mastermix (ABI) for quantitation.

Page

Native polyacrylamide gel electrophoresis (PAGE) of PCR products was performed as described¹⁸. A 10 base-pair (bp) ladder (Invitrogen, Carlsbad, Calif.) was used for sizing PCR products in all experiments (Marker (M)). Typically, 8 μl of each PCR sample was analyzed by PAGE.

Statistical Analysis

Statistical analysis was performed by one-way analysis of variance (ANOVA) followed by Tukey's Honestly Significant Difference (HSD) test where indicated. Column graphed data are presented as +/− one standard deviation of the mean. Data points were compared by the unpaired one-tailed t test unless otherwise indicated, and the P-values are labeled in the figures, text or in the legend, as are the number of independent experiments.

Results

NaF and KOx improve MicroRNA Quantitation

Different anticoagulants and blood stabilizers are used for plasma collection, but serum is collected without stabilizers or anticoagulants. Therefore, we tested whether the choice of collection method affected miRNA quantitation. We collected blood into blood evacuation tubes containing one of the following anticoagulants: EDTA, heparin, sodium citrate (Citrate), sodium fluoride and potassium oxalate (NaF/KOx), or collected blood in the absence of anticoagulants. Blood was collected into the different blood evacuation tubes in immediate succession using a single venipuncture per person (Materials and Methods), to ensure near-identical blood composition at the time of blood collection. Blood was drawn from four individuals of different ethnic origin, sex and age on three separate dates.

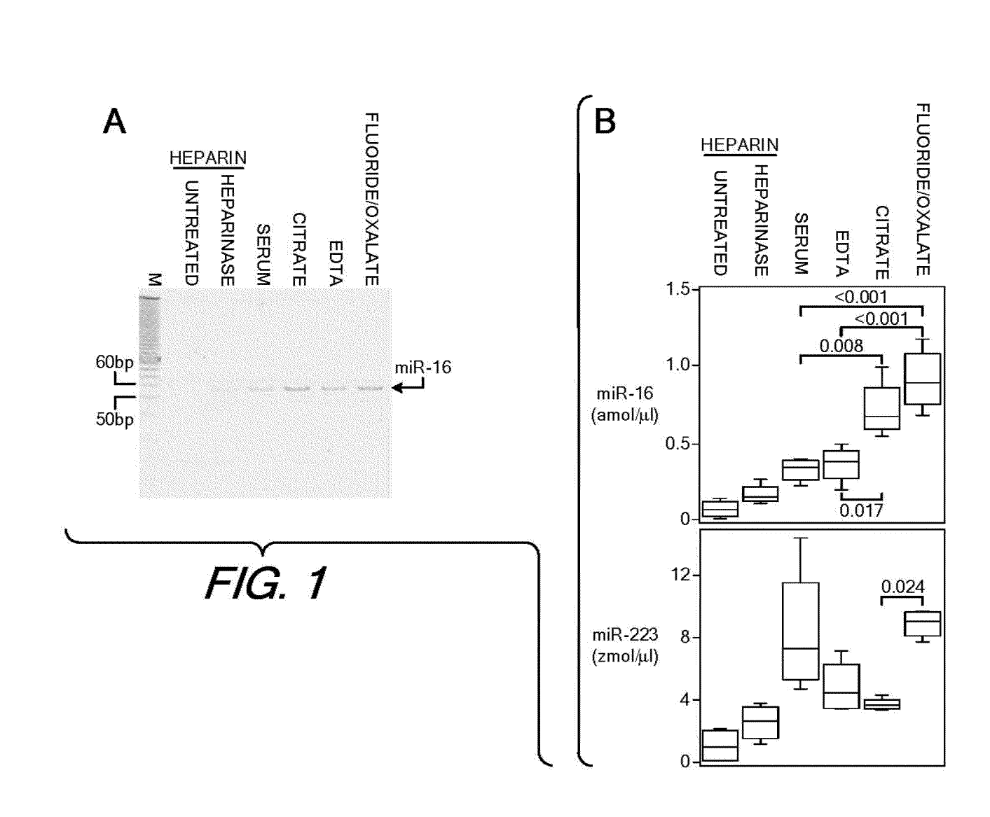

We measured miRNA abundance by reverse-transcription PCR (RT-PCR) (FIG. 1A), and quantitative PCR (qPCR) of reverse-transcribed miRNAs with Sybr Green (FIG. 1B). We found that reproducibility of miRNA quantitation depended on the blood collection methods, with best results obtained by collecting into the tubes containing NaF/KOx (FIG. 1B). Although miR-16 is about 500-fold more abundant than miR-223 in blood plasma, the recovery and accurate detection of both miRNAs depended on the plasma collection method. Therefore, these differences in detection and quantitation resulting from the choice of blood collection methods are likely to occur in measuring other circulating miRNAs, regardless of abundance. Furthermore, quantitation of miR-223 in serum yielded more variable results than most other collection methods, suggesting that at least for some miRNAs, collection of blood as plasma, and the choice of anticoagulants can improve detection.

NaF and KOx Improve the Sensitivity of MicroRNA Detection Post-Blood Collection

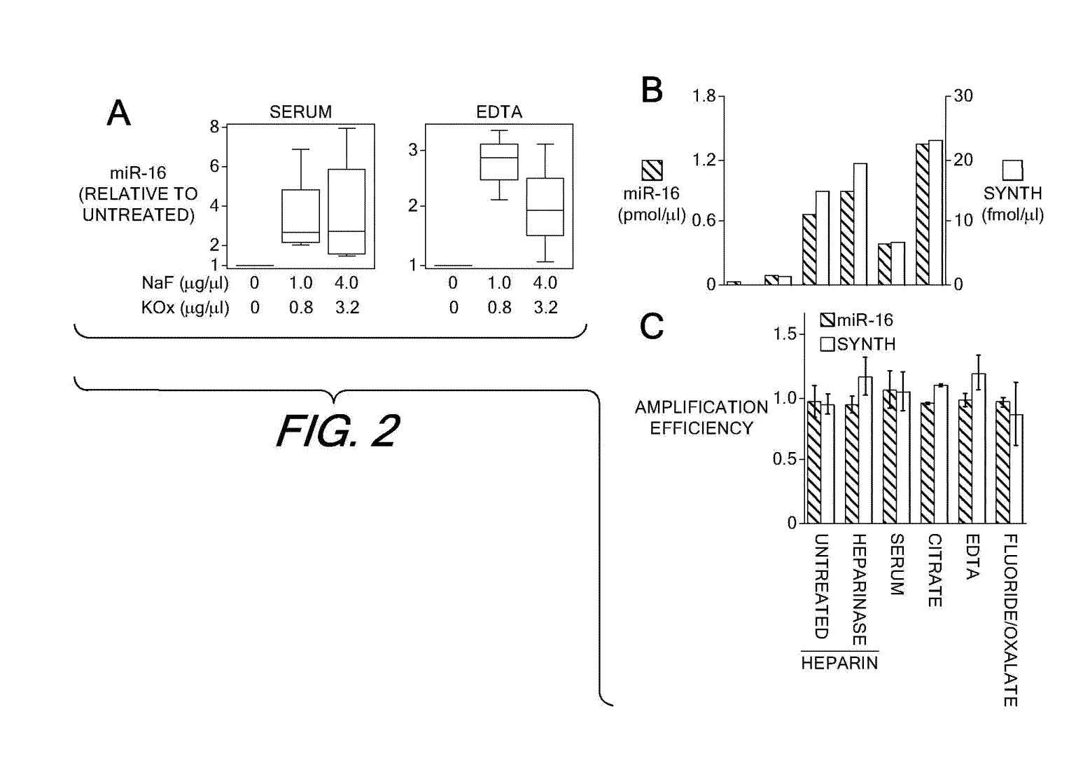

Maintenance of RNA stability during and after blood collection is an important factor for accurate miRNA quantitation and may depend on the anticoagulant used to collect the sample. For example, NaF/KOx is thought to allow greater sensitivity in the quantitation of other molecules in the blood, including glucose²², alcohol and opioids³⁰, by preventing their degradation at the time of collection and during storage³¹. We reasoned that the anticoagulants may differentially affect miRNA stability, or detection by interfering with, or promoting reactions used for quantitation. Therefore, we evaluated the effect that NaF and KOx have on the quantitation of miRNAs collected by other blood draw methods. We added NaF and KOx to frozen samples collected by two of the most common blood draw methods, serum and plasma collected into EDTA, and measured miR-16 in these samples (Materials and Methods). We found that NaF and KOx increased miR-16 detection 2-fold in plasma collected using EDTA (p=0.037) and 3-fold in serum (p=0.014) respectively (FIG. 2A). These results indicate that adding NaF and KOx enhances miRNA quantitation even if these reagents are added after the samples were collected using other anticoagulants and stabilizers.

To address how NaF and KOx improve miRNA quantitation, we assessed the effect that NaF and KOx have individually on miR-16 quantitation in serum. NaF increased the mean detection of miR-16 2.8 fold, and KOx increased sensitivity about 3.4 fold. However, the increases lacked statistical significance for each component alone, suggesting that NaF and KOx synergize to effectively increase detection of miRNAs, or that they act differently in the improvement of quantitation of miRNAs in serum.

NaF and KOx Increase the Detection of Exogenous RNA

We considered that collecting plasma with NaF and KOx can improve detection by 1) stabilizing the RNA, or 2) increasing the amplification efficiency of the miRNAs during the PCR reaction^{29,32}. To address the possibility that miRNAs are degraded by plasma ribonucleases³³, we quantified miRNA stability prior to miRNA extraction by comparing endogenous miR-16 concentrations to that of a synthetic 22 nucleotide RNA (SYNTH) added during the RNA extraction. We found that the quantity of RNA was similar for both SYNTH RNA and the endogenous miR-16 for each of the different blood collection containers (FIG. 2B), suggesting that factors other than RNA stability in serum or plasma account for the differences in the sensitivity of RNA quantitation. We therefore tested whether the collection method affects the PCR amplification efficiency. We detected no significant changes in the amplification efficiencies induced by the blood stabilizers or anti-coagulants (FIG. 2C). We conclude from this result that none of the blood collection methods affect the PCR amplification of reverse-transcribed miRNAs per se. Hence NaF and KOx may improve miRNA detection by enhancing miRNA yield during the extraction, by enhancing the reverse transcriptase reaction, or perhaps in general, by stabilizing the extracted RNA or the cDNA.

The finding of strong parallels between SYNTH and miR-16 quantitation within each plasma and serum sample support the convention of using exogenous spiked RNAs as reference molecules for quantifying miRNA in plasma and serum. Alternatively, we found that quantitation of SYNTH added in a 4000-fold excess was much less affected by most collection methods. This result suggests that high concentrations of RNA can overcome the interference of plasma components on miRNA quantitation, and highlights the need to supply spiked RNA at similar concentrations as the endogenous RNA under investigation for accurate reference quantitation.

Inhibitors of Polymerases Present in Biological Samples Affect MicroRNA Detection

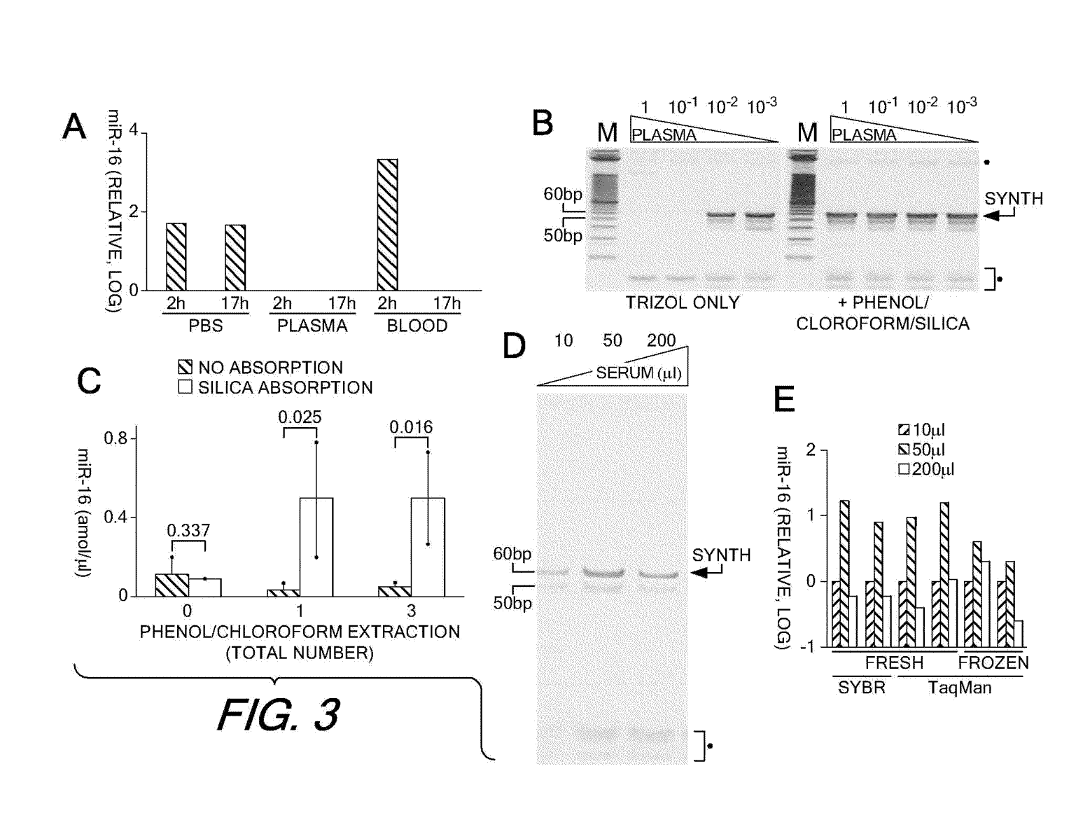

To assess the stability of miRNAs in blood, we added released miRNAs isolated from the media of cultured BC3 cells to PBS, plasma and whole blood. We found that miR-16 could be detected up to 17 h after addition to PBS, but was undetectable in plasma after 2 h or whole blood after 17 hours of storage (FIG. 3A). This destabilization effect could be due to miRNA degradation in the sample³⁴, or because of components of blood plasma that co-purify with miRNAs and interfere with their detection^{29,35-37}, or both.

Therefore, we tested whether plasma RNA interferes with the detection of synthetic SYNTH RNA. To do so, we supplemented SYNTH RNA with Trizol-extracted RNA preparations of plasma collected in EDTA VACUTAINER® blood tubes, and quantified SYNTH RNA abundance (FIG. 3B, left panel). We found that RNA extracted from blood plasma interfered with SYNTH RNA detection, indicating that inhibitors of reverse transcriptase or PCR are present in RNA preparations extracted with Trizol alone.

To determine the best method for removing serum and plasma inhibitors of miRNA detection, we applied approaches to improve the purity of the isolated RNA. We found that the incorporation of a single acidic phenol/chloroform extraction step followed by adsorption of RNA on silica membranes reduced the interference by blood-borne polymerase inhibitors (FIG. 3B, right panel), and effected a 4.4-fold increase in miRNA quantitation (FIG. 3C). Other treatments, such as the addition of detergents³⁸ or ribonuclease inhibitor RNAsin did not improve miRNA detection in serum or plasma suggesting that components other than ribonucleases or other proteins were responsible for the interference. We conclude that enrichment of small RNAs using Trizol and phenol/chloroform extractions and silica-adsorption effectively remove blood-borne RT and/or PCR inhibitors that prevent accurate quantitation of miRNAs present in blood plasma.

### Plasma Volume Affects MiRNA Detection and Quantitation

The presence of inhibitors of miRNA detection in blood suggests that the greater the plasma or serum starting material used to extract the miRNA, the greater the abundance of co-purified blood-borne inhibitors of RT-PCR. Thus, we tested whether dilution of starting material affects the efficiency of detection. To do so, we quantified miRNA extracted from 10 μl, 50 μl or 200 μl of fresh serum or plasma. We found that 50 μl of serum improved detection of miRNA by end-point PCR (FIG. 3D), and yielded an 11-fold increase in the sensitivity of miRNA detection by SYBR Green or TaqMan qPCR (FIG. 3E), perhaps reflecting a balance between miRNA and inhibitor abundance. To test if similar concentration effects also apply to stored samples, we tested plasma collected into EDTA from HIV and KSHV infected patients, and stored for several months. We found that, similar to fresh samples, detection of miR-16 abundance was about 3-fold more sensitive at 50 μl than 10 μl or 200 μl (FIG. 3E). These data are consistent with the counter-intuitive idea that using more blood for detecting miRNA actually results in less efficient detection than using less blood. Because this effect was seen in both plasma and serum, the latter of which is collected in the absence of additives, we concluded that inhibitors of miRNA detection are inherent to blood, rather than introduced by chemicals used for collection. Importantly, by reducing the starting material, inhibitors were presumably diluted below a threshold of interference. Thus, careful titration of starting material yields more accurate miRNA quantitation.

Heparinase Treatment of Plasma Increases microRNA Detection.

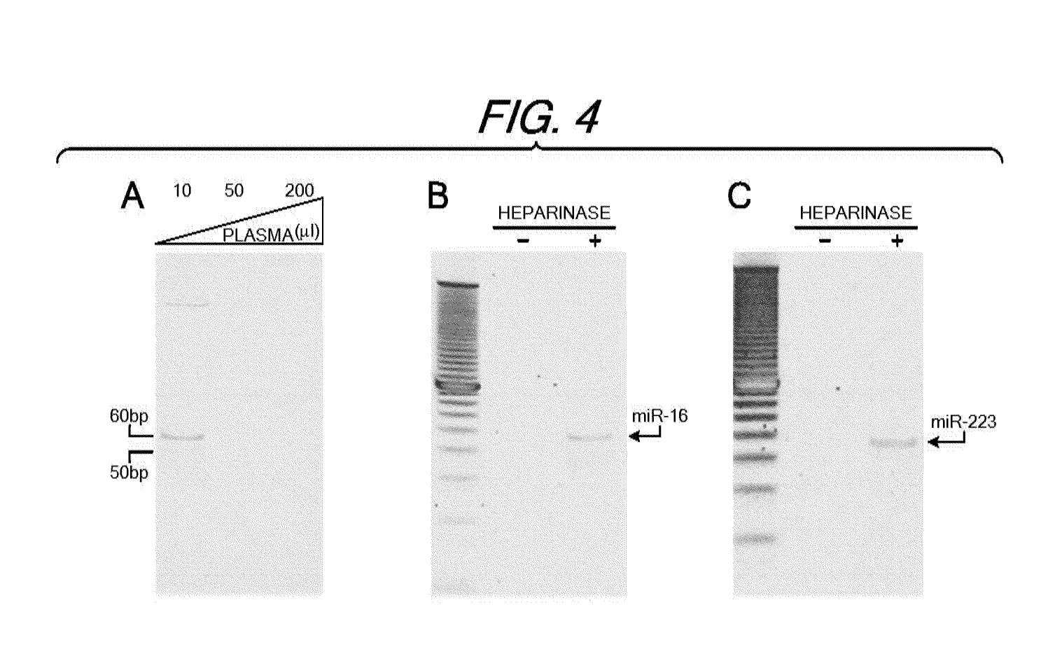

Heparin is an endogenous component of blood, and one of the original anti-coagulants used in medicine. In some instances, for example when analyzing historical samples, or evaluating the blood of patients on heparin regimens used for deep venous thromboses, strokes, pulmonary embolism, during organ transplantation or heart surgery, heparinized plasma may be the only source of miRNA. In these cases, it will be important to evaluate the effect of heparin on miRNA quantitation. We found that miRNA quantified from heparinized plasma gave a poor yield (FIG. 1), consistent with the fact that RT-PCR is inhibited by heparin³⁹. We tested whether reducing the starting volume could improve detection. We found that using less starting material allowed detection of miR-16 from heparinized blood. However, greater dilutions were required to effect similar detection as in plasma collected by other methods (compare FIG. 3D to FIG. 4A). We therefore tested whether miRNA detection can be improved by digesting heparin with lyase heparinase I prior to RT-PCR^{24,40}. Strikingly, this treatment allowed the detection and quantitation of miRNAs that were previously undetectable (FIGS. 4B and 4C). We conclude that heparinase can increase detection of miRNAs in heparinized plasma. Importantly, these results suggest that heparin tubes should be avoided when possible for miRNA analyses. Alternatively, heparinase treatment provides an approach to detect miRNAs in cases where the use of heparin was unavoidable or when heparin is present in previously collected samples.

A Mutant Taq DNA Polymerase Improves Quantitation of MicroRNA.

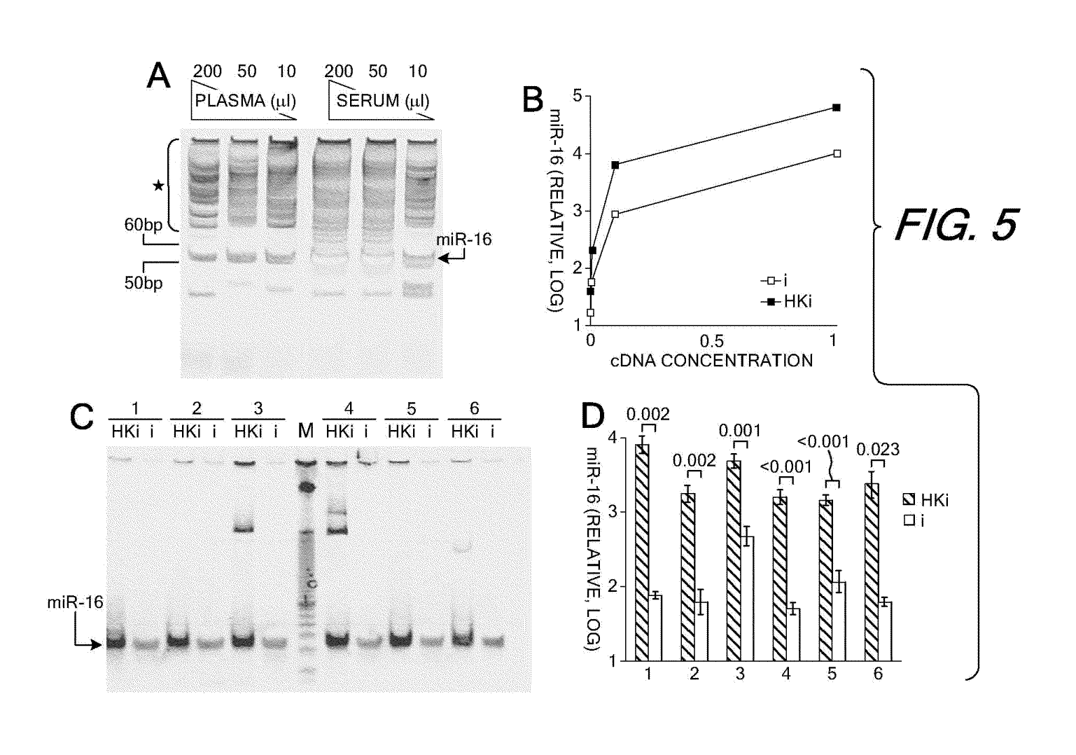

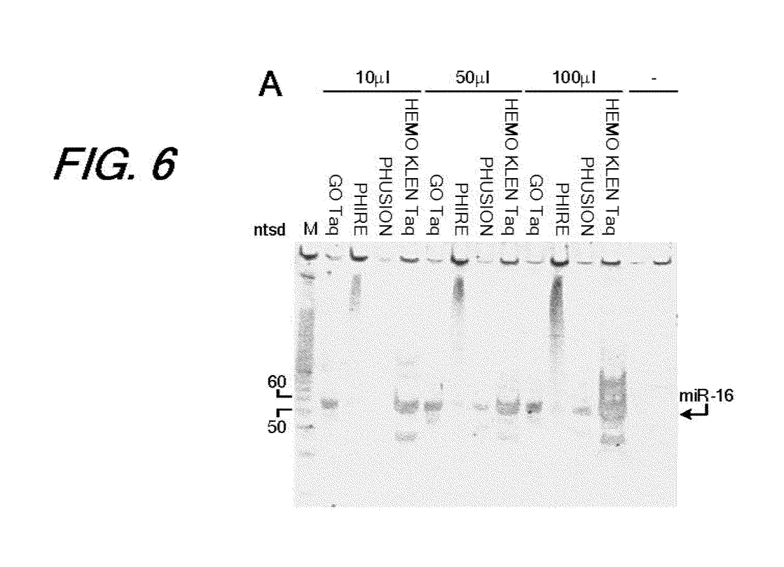

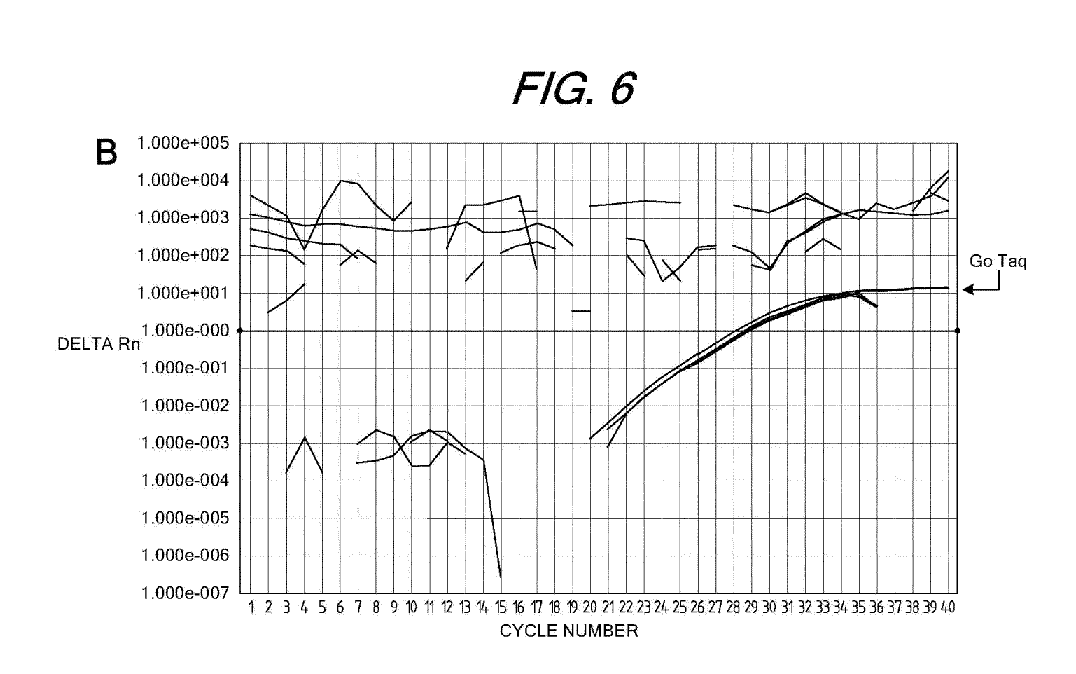

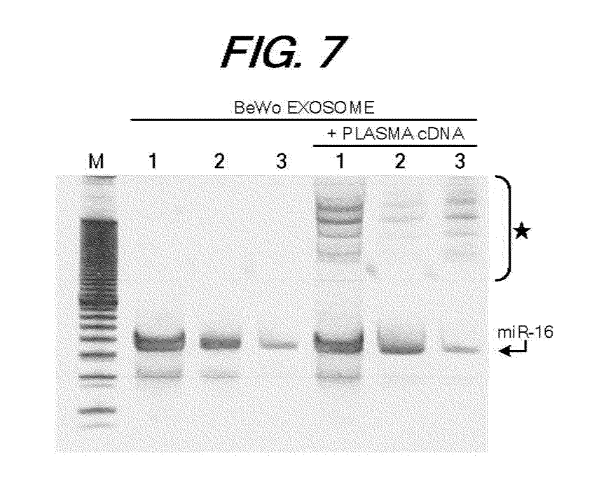

An alternative approach to avoid interference from blood-borne inhibitors of RT-PCR is to use different polymerases^{29,35-37,41}. We tested enzymes reported to be more resistant to inhibitors: Phusion, Phire, and Hemo KlenTaq. Hemo KlenTaq is a mutant Taq DNA polymerase, which reportedly has a 100-fold lower sensitivity to blood inhibitors than wild-type Taq²⁹. Our analysis indicates that Hemo KlenTaq amplified miR-16, more efficiently than Phusion (FIGS. 5A and 6) and standard Taq DNA polymerase yielded low or no detectable PCR products in the same samples (FIG. 3D). However, initial attempts to adopt these enzymes for qPCR of miRNAs using TaqMan failed (FIG. 6B). Likely, for Hemo KlenTaq the quantitation was compromised by the production of multiple spurious bands in addition to the correct band (FIGS. 5A and 6A) We tested if this lack of specificity is a general property of Hemo KlenTaq, by evaluating the purity of amplifying miR-16 released from BeWo cells in culture. We detected only the correct, miR-16 PCR product. However, supplementing such PCR reactions with blood plasma cDNA was sufficient to give rise to the extra products, regardless of attempts to purify cDNA further prior to PCR amplification (FIG. 7). This finding suggests that the complexity or other properties of blood cDNA interfere with the exclusive amplification of the miRNAs of interest and cause non-specific cDNA amplification. This phenomenon may be a consequence of the lack of the editing- and proofreading 5′->3′ exonuclease domain in Hemo KlenTaq³².

A Complementing Enzyme Cocktail for Effective Amplification of Products

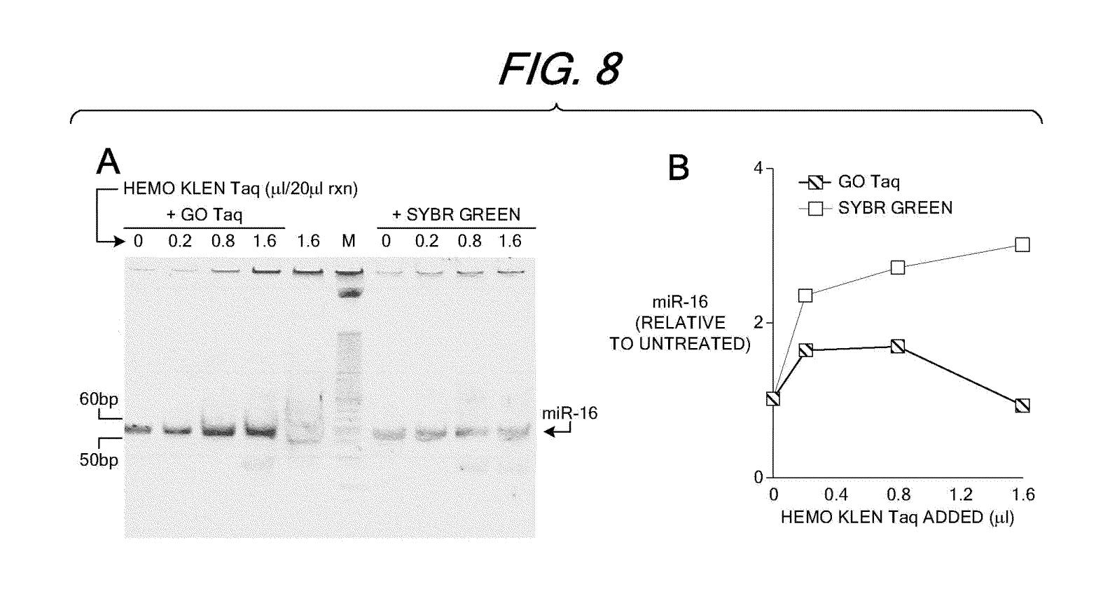

To overcome the limitations of Hemo KlenTaq, we tested whether the reduced proof-reading activity of Hemo KlenTaq could be complemented by an intact Taq polymerase. We found that Hemo KlenTaq in combination with intact Taq polymerases amplified specific PCR products suitable for qPCR by SYBR Green or TaqMan (FIGS. 5B, 5C and 8A). Importantly, the complementing Taq polymerases produced about 30 times more amplification products from plasma miR-16 on average than the intact polymerase alone (FIG. 8B) suggesting that overcoming blood-borne inhibitors of Taq polymerase using Hemo KlenTaq yield an overall increase in detection sensitivity and specificity of circulating miRNAs.

Mir-16 Quantitation Sensitive to Method of Detection

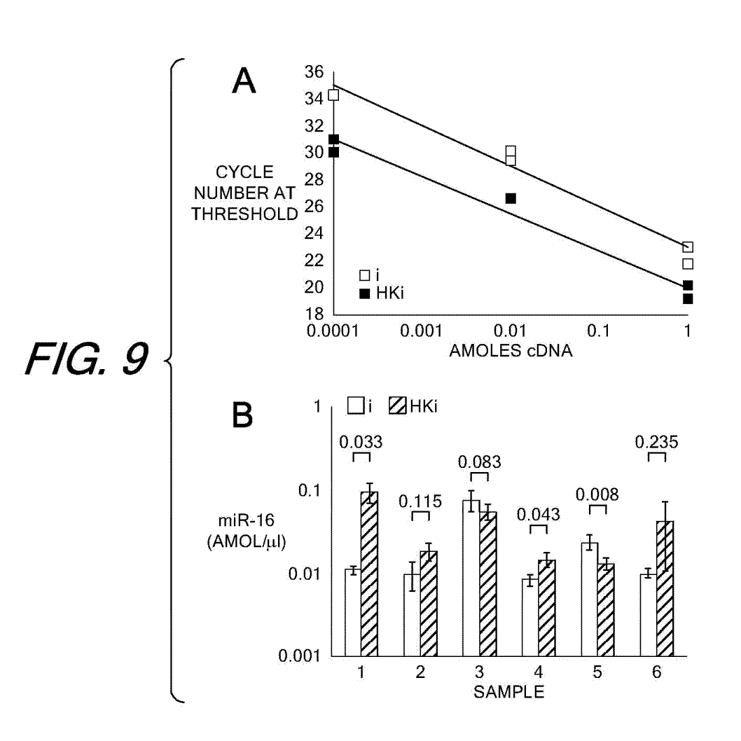

To test if overcoming endogenous inhibitors with complementing polymerases provides proportionally higher miRNA quantitation, we analyzed circulating miRNAs in the blood of six healthy individuals (FIGS. 9A,B) Five of the six individuals tested show similar miR-16 plasma concentrations, regardless of approach used, thus validating the use of complementing enzymes for improved detection of circulating miRNAs. Interestingly, one individual in each approach consistently had higher plasma miR-16 abundance than the others (FIG. 5D). However, a different individual in either approach had higher miR-16 concentrations, suggesting that the quantitation of circulating miR-16, which is used as a reference miRNA or a biomarker of some cancers¹⁶⁻¹⁹, depends on the PCR conditions. These results suggest that differences in plasma composition among individual donors can yield different miRNA measurements.

TABLE 1
Effectiveness in MiRNA Detection With Different Treatments

| | | | | | |
|---|---|---|---|---|---|
| Treatment/ | | Extra | | Sensitivity/ | |
| Approach | Sensitivity¹ | Time² | Cost³ | Cost⁴ | FIGS. |

Choice of anticoagulant and stabilizer

| | | | | | |
|---|---|---|---|---|---|
| NaF/KOx | 2.7 | 0 | 99% | 2.7 | 1 |
| EDTA | 1.1 | 0 | 99% | 1.1 | 1 |
| Citrate | 1.7 | 0 | 100% | 1.7 | 1 |
| Heparin | 0.3 | 0 | 101% | 0.3 | 1 |
| Serum | 1 | 0 | 100% | 1.0 | 1 |
| NaF/KOx post- | 3.6⁵ | seconds | 100% | 3.6 | 2A |
| collection | | | | | |

Starting volume

| | | | | | |
|---|---|---|---|---|---|
| 10 μl | 1 | 0 | 100% | 1.0 | 3D, E, |
| | | | | | 4A |
| 50 μl | 11⁶ | 0 | 100% | 11 | 3D, E, |
| | | | | | 4A |
| 200 μl | 0.7 | 0 | 100% | 0.7 | 3D, E, |
| | | | | | 4A |
| Silica/Phenol/ | 4.4 | 11 min⁷ | 181% | 2.4 | 3B, C |
| Chloroform | | | | | |
| Hemo Klentaq/Go | 30 | seconds | 101% | 30 | 5 |
| Taq | | | | | |
| Heparinase | 2⁸ | 1 h | 130% | 1.5 | 1, 4 |
| treatment | | | | | |

¹Approximate fold difference in detected abundance compared to serum miR-16.
²Per sample.
³Material cost per sample: Calculated as % of total cost of serum miR-16 detection using TaqMan (in $, based on list prices of material used within study, which was $9.25 for triplicate TaqMan)
⁴Increase in Sensitivity returned per Material investment.
⁵2.4-fold in EDTA plasma.
⁶3-fold in frozen samples.
⁷the extra total time spent on adsorption per sample is gained by elimination of the chilling step for precipitation of RNA (1 h plus) in the standard method. However, there is an increase in active labor, which, depending on the number of samples processed, may by substantial.
⁸Fold of plasma collected in heparin. Ranges from statistically non-significant changes (FIG. 1) to detecting signal only after heparinase treatment (FIG. 4).

Discussion

This study demonstrates that inherent differences in biological samples and the methods used to collect and analyze them can dramatically affect the detection and quantitation of miRNAs. The implications of the work are that without consideration of the variables we have identified, miRNA quantitation from human samples may not be reliable for the purpose of biomarker development. Our results suggest that failure to detect plasma miRNAs may be due to polymerase inhibitors rather than the actual absence of miRNA. Such inhibitors may include hemoglobin⁴², lactoferrin³⁶, and immunoglobulin G⁴³, which can co-purify with nucleic acids³⁷. This limitation can be overcome by the concomitant use of two complementing Taq polymerases; Hemo KlenTaq, which is resistant to blood-borne inhibitors, in combination with another intact polymerase that has effective proof-reading ability. We also demonstrate that diluting out inhibitors from blood samples also provides salient improvements in miRNA detection (Table 1). Additional purification of plasma or serum miRNA preparations, using organic extraction and silica adsorption to remove inhibitors, also increased the detection, albeit at a greater cost in labor and funds.

The inability to detect specific miRNAs in plasma or serum, in many cases, reflects the low abundance of particular miRNAs in the circulation. For example, the release of some miRNAs from cells into blood is limited or selective^{18,44,45}. Furthermore, depending on the nature of the complex circulating miRNAs are associated with^{18,34,46-52}, some miRNAs may be more stable than others³⁴ to the degradation by plasma ribonucleases, or may be more amenable to amplification by polymerases than others. The improvements outlined here allow the quantitation of miRNAs with very low abundance, where usual techniques fail. The use of these approaches is expected to increase the repertoire of miRNAs that can be analyzed as potential biomarkers of disease.

The mechanism by which some of the approaches, for example the use NaF/KOx improve detection is unclear. Possibly, NaF effects higher cDNA stability during the reverse transcriptase reaction, rather than stabilize input miRNA, because classical experiments identified NaF as an inhibitor of RNAse H⁵³, an enzyme that degrades RNA/DNA hybrid substrates. On the other hand, KOx may promote miRNA detection by reducing calcium, a known inhibitor of Taq, in the blood sample⁴¹.

Interestingly, the absolute quantity of circulating miR-16 measured in some individuals' blood was different depending on the enzyme used. These differences raise the possibility that factors, including diet⁵⁴, exercise⁵⁵, circadian rhythms⁵⁶ and seasons^{57,58}, which alter the blood chemistry might ultimately affect miRNA detection and quantitation. The disparate effectiveness of miRNA detection in individual's plasma or sera may be indicative of other, physiologic and pathogenic properties of the blood, including endogenous heparin concentrations⁵⁹ or familial disease⁶⁰. Our results demonstrate that such variations in blood chemistry can affect the detection of miRNAs, and must be considered and neutralized in order to accurately and efficiently assess miRNA abundance in blood serum and plasma.

From the foregoing, it will be observed that numerous variations and modifications may be effected without departing from the spirit and scope of the invention. It is to be understood that no limitation with respect to the specific apparatus illustrated herein is intended or should be inferred. It is, of course, intended to cover by the appended claims all such modifications as fall within the scope of the claims

What is claimed is:

1. A kit for measuring the content of micro-RNA in a human blood sample comprising:
a blood collection tube containing at least 1 μg/μl NaF and 0.8 μg/μl KOx; and
a set of instructions for collecting a human blood sample in the blood collection tube.

2. The kit of claim 1 wherein the blood collection tube is free from heparin.

3. A kit for measuring the content of micro-RNA in a human blood sample comprising:
a blood collection tube;
a quantity of NaF and KOx for adding to the blood collection tube so that the collection tube has at least 1 μg/μl NaF and 0.8 μg/μl KOx; and
a set of instructions for collecting human blood sample in the collection tube.

4. The kit of claim 3 further comprising:
a quantity of heparinase I for adding to the blood collection tube.

5. A method for collecting blood from a human subject comprising the steps of:
providing a sample of human blood in a blood collection tube;
adding a quantity of NaF and KOx to the blood collection tube so that the blood collection tube has at least 1 μg/μl NaF and 0.8 μg/μl KOx;
extracting miRNA from the blood collection tube; and
quantifying the miRNA in the sample.

6. The method of claim 5 wherein the human blood has a volume of 5 ml.

7. The method of claim 5 wherein the blood sample is whole blood, serum or plasma.

8. The method of claim 7 wherein the step of quantifying the miRNA is through a polymerase chain reaction procedure.

9. The method of claim 8 wherein the polymerase chain reaction procedure utilizes Hemo KlenTaq and an intact Taq polymerase.

10. The method of claim 5 further comprising the step of adding heparinase Ito the collection tube.
